(19) United States
(12) Patent Application Publication
(10) Pub. No.: US 2019/0154676 A1
(43) Pub. Date: May 23, 2019

(54) DETECTION SYSTEM, DETECTION APPARATUS, AND DETECTION METHOD

(71) Applicant: TDK CORPORATION, Tokyo (JP)

(72) Inventors: Haruki YUGA, Tokyo (JP); Seigo ZAIMA, Tokyo (JP); Takashi KIKUKAWA, Tokyo (JP); Sachio TSUBOIKE, Tokyo (JP)

(73) Assignee: TDK CORPORATION, Tokyo (JP)

(21) Appl. No.: 15/752,210

(22) PCT Filed: Jul. 22, 2016
(86) PCT No.: PCT/JP2016/071528

§ 371 Date: Feb. 12, 2018

(30) Foreign Application Priority Data
Aug. 19, 2015 (JP) 2015-161526

(51) Int. Cl. G01N 33/543

(52) U.S. Cl. CPC G01N 33/54333; G01N 2446/00; G01N 2458/00

(57) ABSTRACT

A detection system includes a detection device including a space, defined by a wall surface, into which a liquid containing a biomolecular label and a liquid containing magnetic beads are introduced and a magnetic sensor having a surface forming a portion of the wall surface, wherein at least some of the magnetic beads bind to the biomolecular label immobilized on the surface of the magnetic sensor or to a molecule near the biomolecular label immobilized on the surface of the magnetic sensor; and a first magnetic-field applying mechanism that applies a magnetic field in a direction in which the magnetic beads are moved away from the surface of the magnetic sensor.

### TECHNICAL FIELD

The present invention relates to techniques for detecting biomolecular labels using magnetic beads and to detection systems, detection apparatuses, and detection methods.

### BACKGROUND ART

Recently, the identification, prevention, and diagnosis of diseases based on quantitative digital data, such as electrical signals, have become increasingly possible with the advances in medical engineering. In practice, digital detection of various proteins, bacteria, viruses, deoxyribonucleic acid (DNA), and ribonucleic acid (RNA), which are biologically derived, has been performed by a combination of immunological and engineering techniques and has been utilized for the identification, prevention, and diagnosis of diseases such as cancer.

Proteins such as cancer markers and hormones are present in vivo in extremely small amounts; therefore, high sensitivity is required for their detection. Furthermore, smaller volumes of samples have been taken in order to reduce the burden on patients. For measurements such as blood electrolyte measurements, detection has already been performed with several microliters of samples.

One approach to achieve high sensitivity is to use magnetic biosensors. Magnetic biosensors, which are a type of sensitive sensing system that has recently been proposed, determine the presence and number of magnetic beads located near the surface of a detection section and thereby determine the presence and concentration of biomolecular labels such as proteins, bacteria, viruses, DNA, and RNA in sample solutions.

PTL 1 discloses a biomolecular label detector capable of sensitive detection of biomolecular labels using a channel through which a sample solution containing a biomolecular label and magnetic beads flows and a magnetic sensor serving as a detection section. The surface of the magnetic sensor forms a portion of the channel wall surface. As an external magnetic field is applied to magnetic beads bound to a biomolecular label immobilized on the surface of the magnetic sensor, a leakage magnetic field is detected by the magnetic sensor to measure the quantity of biomolecular label.

### CITATION LIST

### Patent Literature

PTL 1: Japanese Unexamined Patent Application Publication No. 2013-029440

### SUMMARY OF INVENTION

### Technical Problem

Some of the magnetic beads flowing through the channel do not bind to the biomolecular label. Some of these magnetic beads are adsorbed on the channel wall surface without particular chemical bonding. This adsorption is termed nonspecific adsorption. If nonspecifically adsorbed magnetic beads remain on the surface of the magnetic sensor forming a portion of the channel wall surface, the nonspecifically adsorbed magnetic beads would decrease the accuracy of quantitative detection of the biomolecular label. An object of the present invention is to perform accurate detection of biomolecular labels using magnetic beads.

### Solution to Problem

To achieve the foregoing object, a detection system according to the present invention includes a detection device including a space, defined by a wall surface, into which a liquid containing magnetic beads is introduced and a magnetic sensor having a surface, forming a portion of the wall surface, onto which a biomolecular label is immobilized, wherein at least some of the magnetic beads bind to the biomolecular label or to a molecule near the biomolecular label; and a first magnetic-field applying mechanism that applies a magnetic field in a direction in which the magnetic beads are moved away from the surface of the magnetic sensor.

In the present invention, “magnetic sensor” refers to a sensor that magnetically detects a biomolecular label immobilized on the surface thereof. That is, in the present invention, “the surface of the magnetic sensor” refers to the outermost surface of a layer formed on a magnetic sensing element, i.e., the surface onto which the biomolecular label is immobilized.

In the detection system described above, magnetic beads nonspecifically adsorbed on the surface of the magnetic sensor without being bound to the biomolecular label or to molecules near the biomolecular label are moved away from the surface of the magnetic sensor by the magnetic field applied by the first magnetic-field applying mechanism. This reduces the influence of the nonspecifically adsorbed magnetic beads during magnetic bead detection using the magnetic sensor, thus permitting accurate detection of the magnetic beads bound to the biomolecular label or to the molecules near the biomolecular label.

Furthermore, a detection system according to the present invention includes a detection device including a space, defined by a wall surface, into which a liquid containing a biomolecular label and a liquid containing magnetic beads are introduced and a magnetic sensor having a surface forming a portion of the wall surface, wherein at least some of the magnetic beads bind to the biomolecular label immobilized on the surface of the magnetic sensor or to a molecule near the biomolecular label immobilized on the surface of the magnetic sensor; and a first magnetic-field applying mechanism that applies a magnetic field in a direction in which the magnetic beads are moved away from the surface of the magnetic sensor.

In the detection system described above, magnetic beads nonspecifically adsorbed on the surface of the magnetic sensor without being bound to the biomolecular label or to molecules near the biomolecular label are moved away from the surface of the magnetic sensor by the magnetic field applied by the first magnetic-field applying mechanism. This reduces the influence of the nonspecifically adsorbed magnetic beads during magnetic bead detection using the magnetic sensor, thus permitting accurate detection of the magnetic beads bound to the biomolecular label or to the molecules near the biomolecular label.

Furthermore, the detection system according to the present invention further includes a second magnetic-field applying mechanism that applies a magnetic field to the magnetic beads bound to the biomolecular label or to the molecule near the biomolecular label during magnetic detection using the magnetic sensor.

According to this, the second magnetic-field applying mechanism can be used to apply a magnetic field suitable for magnetic bead detection using the magnetic sensor, thus permitting sensitive detection of the magnetic beads bound to the biomolecular label or to the molecules near the biomolecular label.

Furthermore, in the detection system according to the present invention, the space is a channel space through which the liquid containing the magnetic beads flows, and the surface of the magnetic sensor forms a portion of a channel wall surface defining the channel space.

Furthermore, in the detection system according to the present invention, the space is a channel space through which the liquid containing the biomolecular label and the liquid containing the magnetic beads flow, and the surface of the magnetic sensor forms a portion of a channel wall surface defining the channel space.

To achieve the foregoing object, a detection apparatus according to the present invention includes an insertion section into which a detection device is inserted, the detection device including a space, defined by a wall surface, into which a liquid containing magnetic beads is introduced and a magnetic sensor having a surface, forming a portion of the wall surface, onto which a biomolecular label is immobilized, wherein at least some of the magnetic beads bind to the biomolecular label or to a molecule near the biomolecular label; and a first magnetic-field applying mechanism that applies a magnetic field in a direction in which the magnetic beads are moved away from the surface of the magnetic sensor.

According to this, magnetic beads nonspecifically adsorbed on the surface of the magnetic sensor without being bound to the biomolecular label or to molecules near the biomolecular label are moved away from the surface of the magnetic sensor by the magnetic field applied by the first magnetic-field applying mechanism. This reduces the influence of the nonspecifically adsorbed magnetic beads during magnetic bead detection using the magnetic sensor, thus permitting accurate detection of the magnetic beads bound to the biomolecular label or to the molecules near the biomolecular label.

Furthermore, a detection apparatus according to the present invention includes an insertion section into which a detection device is inserted, the detection device including a space, defined by a wall surface, into which a liquid containing a biomolecular label and a liquid containing magnetic beads are introduced and a magnetic sensor having a surface forming a portion of the wall surface, wherein at least some of the magnetic beads bind to the biomolecular label immobilized on the surface of the magnetic sensor or to a molecule near the biomolecular label immobilized on the surface of the magnetic sensor; and a first magnetic-field applying mechanism that applies a magnetic field in a direction in which the magnetic beads are moved away from the surface of the magnetic sensor.

According to this, magnetic beads nonspecifically adsorbed on the surface of the magnetic sensor without being bound to the biomolecular label or to molecules near the biomolecular label are moved away from the surface of the magnetic sensor by the magnetic field applied by the first magnetic-field applying mechanism. This reduces the influence of the nonspecifically adsorbed magnetic beads during magnetic bead detection using the magnetic sensor, thus permitting accurate detection of the magnetic beads bound to the biomolecular label or to the molecules near the biomolecular label.

Furthermore, the detection apparatuses according to the present invention further include a second magnetic-field applying mechanism that applies a magnetic field to the magnetic beads bound to the biomolecular label or to the molecule near the biomolecular label during magnetic detection using the magnetic sensor.

According to this, the second magnetic-field applying mechanism can be used to apply a magnetic field suitable for magnetic bead detection using the magnetic sensor, thus permitting sensitive detection of the magnetic beads bound to the biomolecular label or to the molecules near the biomolecular label.

Furthermore, in the detection apparatus according to the present invention, the space is a channel space through which the liquid containing the magnetic beads flows, and the surface of the magnetic sensor forms a portion of a channel wall surface defining the channel space.

Furthermore, in the detection apparatus according to the present invention, the space is a channel space through which the liquid containing the biomolecular label and the liquid containing the magnetic beads flow, and the surface of the magnetic sensor forms a portion of a channel wall surface defining the channel space.

To achieve the foregoing object, a detection method according to the present invention includes bringing a liquid containing a biomolecular label into contact with a surface of a magnetic sensor to immobilize the biomolecular label onto the surface of the magnetic sensor; bringing a liquid containing magnetic beads into contact with the surface of the magnetic sensor to bind at least some of the magnetic beads to the biomolecular label immobilized on the surface of the magnetic sensor or to a molecule near the biomolecular label immobilized on the surface of the magnetic sensor; applying a magnetic field in a direction in which the magnetic beads are moved away from the surface of the magnetic sensor; and then performing magnetic detection using the magnetic sensor.

According to this, magnetic beads nonspecifically adsorbed on the surface of the magnetic sensor without being bound to the biomolecular label or to molecules near the biomolecular label are moved away from the surface of the magnetic sensor by the magnetic field applied in the direction in which the magnetic beads are moved away from the surface of the magnetic sensor. This reduces the influence of the nonspecifically adsorbed magnetic beads during magnetic bead detection using the magnetic sensor, thus permitting accurate detection of the magnetic beads bound to the biomolecular label or to the molecules near the biomolecular label.

Furthermore, in the detection method according to the present invention, the liquid containing the biomolecular label is passed through a channel space in which the surface of the magnetic sensor forms a portion of a channel wall surface thereof to immobilize the biomolecular label onto the surface of the magnetic sensor, and the liquid containing the magnetic beads is passed through the channel space to bind at least some of the magnetic beads to the biomolecular label immobilized on the surface of the magnetic sensor or to a molecule near the biomolecular label immobilized on the surface of the magnetic sensor.

Furthermore, in the detection method according to the present invention, a liquid containing no magnetic beads is passed through the channel space while the magnetic field is being applied or after the application of the magnetic field is stopped, and magnetic detection is then performed using the magnetic sensor.

According to this, the liquid containing no magnetic beads is passed through the channel space while a magnetic field is being applied in the direction in which the magnetic beads are moved away from the surface of the magnetic sensor or after the application of the magnetic field is stopped. This allows the nonspecifically adsorbed magnetic beads moved away from the surface of the magnetic sensor to be removed from around the magnetic sensor, thus permitting more accurate detection.

Furthermore, in the detection method according to the present invention, the liquid containing the biomolecular label is introduced into a well space in which the surface of the magnetic sensor forms a portion of a wall surface thereof to immobilize the biomolecular label onto the surface of the magnetic sensor, the liquid containing the magnetic beads is introduced into the well space to bind at least some of the magnetic beads to the biomolecular label immobilized on the surface of the magnetic sensor or to a molecule near the biomolecular label immobilized on the surface of the magnetic sensor, the magnetic field is applied to cause the magnetic beads to become attached to a magnetic-field applying mechanism that applies the magnetic field, and magnetic detection is then performed using the magnetic sensor.

According to this, by causing the magnetic beads to become attached to the first magnetic-field applying mechanism, the nonspecifically adsorbed magnetic beads moved away from the surface of the magnetic sensor can be removed from around the magnetic sensor, thus permitting more accurate detection.

### Advantageous Effects of Invention

The detection systems, the detection apparatuses, and the detection method according to the present invention permit accurate detection of biomolecular labels using magnetic beads.

### BRIEF DESCRIPTION OF DRAWINGS

FIG. 1 is a schematic view of a detection apparatus according to a first embodiment.

FIG. 2 is a schematic view of a detection system according to the first embodiment.

FIG. 3 is a sectional view of a detection device according to the first embodiment taken in a plane perpendicular to a direction in which liquid flows through a channel space.

FIG. 4 is a sectional view taken along line A-A in FIG. 3.

FIG. 5 is a schematic view showing the initial state in the step of pumping a liquid containing a biomolecular label in the first embodiment.

FIG. 6 is a schematic view showing the step of allowing the biomolecular label to settle in the first embodiment.

FIG. 7 is a schematic view showing the step of removing any unimmobilized biomolecular label in the first embodiment.

FIG. 8 is a schematic view showing the initial state in the step of pumping a liquid containing magnetic beads in the first embodiment.

FIG. 9 is a schematic view showing the step of allowing the magnetic beads to settle in the first embodiment.

FIG. 10 is a schematic view showing the step of applying a magnetic field from a first magnetic-field applying mechanism in the first embodiment.

FIG. 11 is a schematic view showing the step of removing nonspecifically adsorbed magnetic beads with a liquid containing no magnetic beads in the first embodiment.

FIG. 12 is a schematic view showing the state after the removal of the nonspecifically adsorbed magnetic beads in the first embodiment.

FIG. 13 is a schematic view showing the state in which the magnetic beads are bound to molecules present on the surface of a magnetic sensor near the biomolecular label.

FIG. 14 is a schematic view showing the state in which the magnetic beads are bound to magnetic-bead supporting molecules selectively grown near the biomolecular label.

FIG. 15 is a schematic view of a detection apparatus according to a second embodiment.

FIG. 16 is a schematic view of a detection system according to the second embodiment.

FIG. 17 is a sectional view of a detection device according to the second embodiment.

FIG. 18 is a schematic view showing the initial state in the step of introducing a liquid containing a biomolecular label in the second embodiment.

FIG. 19 is a schematic view showing the step of allowing the biomolecular label to settle in the second embodiment.

FIG. 20 is a schematic view showing the step of removing any unimmobilized biomolecular label in the second embodiment.

FIG. 21 is a schematic view showing the initial state in the step of introducing a liquid containing magnetic beads in the second embodiment.

FIG. 22 is a schematic view showing the step of allowing the magnetic beads to settle in the second embodiment.

FIG. 23 is a schematic view showing the step of applying a magnetic field from a first magnetic-field applying mechanism in the second embodiment.

FIG. 24 is a schematic view showing the state in which the magnetic beads are attached to the first magnetic-field applying mechanism in the second embodiment.

FIG. 25 is a schematic view showing the state after the removal of the nonspecifically adsorbed magnetic beads in the second embodiment.

FIG. 26 is a schematic view showing the state in which the magnetic beads are removed with a dropper in a modification of the second embodiment.

FIG. 27 is a sectional view of a detection device according to another modification of the second embodiment.

FIG. 28 is a sectional view of a detection device according to still another modification of the second embodiment.

FIG. 29 is a sectional view of a detection device according to still another modification of the second embodiment.

### DESCRIPTION OF EMBODIMENTS

Example embodiments of the present invention will now be described with reference to the drawings. The following description is intended to illustrate some embodiments of the invention. The invention is not limited to these embodiments; any embodiment that possesses the technical idea of the invention is included in the scope of the invention. For example, the various configurations in the various embodiments and combinations thereof are given by way of example, and additions, deletions, substitutions, and other modifications can be made to these configurations without departing from the spirit of the invention.

### First Embodiment

### Detection Apparatus

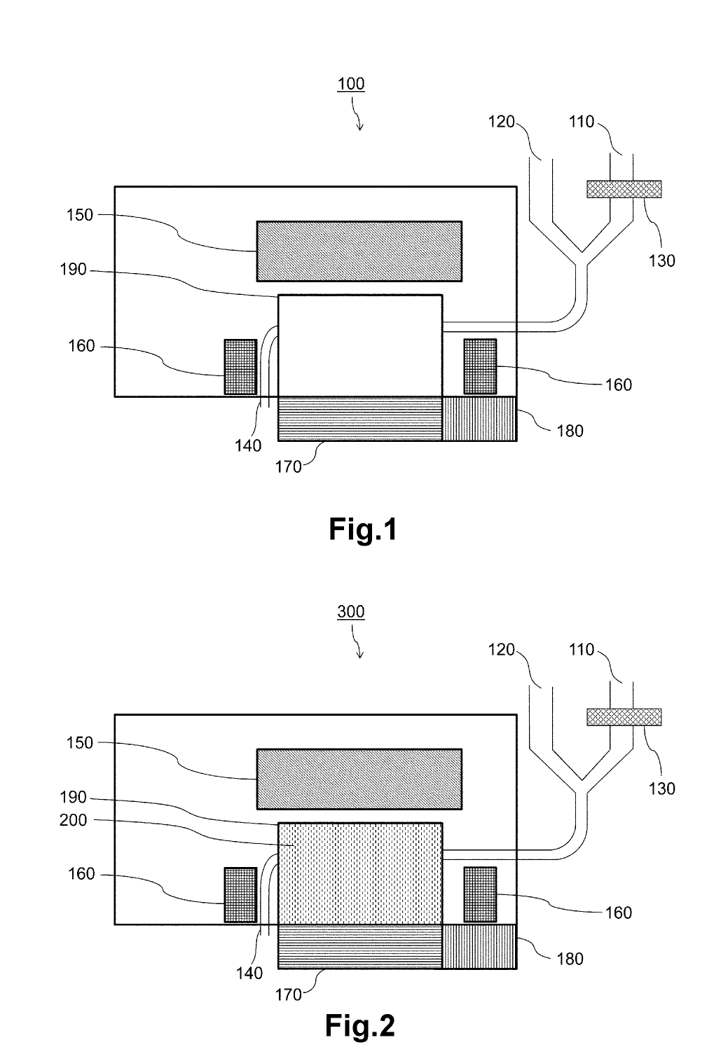

As shown in FIG. 1, a detection apparatus 100 according to a first embodiment includes a biologically-derived-liquid inlet 110, a magnetic-bead containing liquid inlet 120, a bodily-fluid chemical treatment section 130, a liquid outlet 140, a first magnetic-field applying mechanism 150, a second magnetic-field applying mechanism 160, an electrical-signal converting section 170, a display section 180, and a detection device insertion slot 190 (insertion section into which a detection device 200, described later, is inserted).

### Detection System

As shown in FIG. 2, a detection system 300 according to the first embodiment includes the detection apparatus 100 and the detection device 200, described later. The detection system 300 operates with the detection device 200 inserted in the detection device insertion slot 190 of the detection apparatus 100.

The biologically-derived-liquid inlet 110 is an inlet through which a biologically derived liquid is supplied. The biologically-derived-liquid inlet 110 is formed of a material selected according to need, such as metal, plastic, resin, or glass. Examples of biologically derived liquids that may be supplied include bodily fluids and biomolecular-label containing liquids. Examples of bodily fluids include blood, serum, oral washings, urine, cerebrospinal fluid, sputum, biopsy samples, and bone marrow samples. The bodily fluid is treated in the bodily-fluid chemical treatment section 130, described later. The bodily fluid to be supplied to the detection apparatus 100 may be treated in advance to supply a liquid containing molecules converted into a biomolecular label (biomolecular-label containing liquid) from the biologically-derived-liquid inlet 110. In this case, the biomolecular-label containing liquid need not be passed through the bodily-fluid chemical treatment section 130.

The magnetic-bead containing liquid inlet 120 is an inlet through which a liquid containing magnetic beads 410, described later, is supplied. The magnetic-bead containing liquid inlet 120 is formed of a material selected according to need, such as metal, plastic, resin, or glass. The liquid containing the magnetic beads 410 needs to have the property of well dispersing the magnetic beads 410 without dissolving them. A buffer solution having a suitable pH is selected depending on the type of binding reaction between the magnetic beads 410 and a biomolecular label 400. For example, a buffer solution containing trishydroxymethylaminomethane and ethylenediaminetetraacetic acid and adjusted to a pH of 7 to 8 is used for the binding of biotin, serving as the biomolecular label 400, to streptavidin, serving as a surface substance on the magnetic beads 410.

The bodily-fluid chemical treatment section 130 is a section in which, if the liquid supplied from the biologically-derived-liquid inlet 110 is a bodily fluid, for example, cells, proteins, or analyte molecules present in the bodily fluid are converted into a biomolecular label by chemical treatment. Specifically, this chemical treatment involves processes such as lysing cells present in the bodily fluid, extracting, for example, a nucleic acid, protein, or analyte molecules, capturing the nucleic acid, protein, or analyte molecules with, for example, an antibody or nucleic acid, cleaving the antibody or nucleic acid with a particular restriction enzyme, and binding label molecules to the antibody or nucleic acid. Suitable substances are selected for capturing, cleaving, and labeling depending on the disease to be identified, prevented, or diagnosed.

The liquids supplied to the biologically-derived-liquid inlet 110 and the magnetic-bead containing liquid inlet 120 flow into a channel space 230 in the detection device 200, described later.

The liquid outlet 140 is an outlet through which liquid is drained from the channel space 230 in the detection device 200. The liquid outlet 140 is formed of a material selected according to need, such as metal, plastic, resin, or glass. A pump is preferably used at an appropriate position in the detection apparatus 100 to allow liquid to be supplied and drained by efficient pumping.

The first magnetic-field applying mechanism 150 is a mechanism that applies a magnetic field in a direction in which the magnetic beads 410 in the detection device 200, described later, are moved away from the surface of a magnetic sensor 220, described later. The first magnetic-field applying mechanism 150 may be any member having a mechanism or function that allows it to apply a magnetic field, such as a permanent magnet or coil (electromagnet). A coil (electromagnet) is preferably used since the applied magnetic field can be electrically controlled and the apparatus configuration can be simplified.

The second magnetic-field applying mechanism 160 is a mechanism for applying a magnetic field required for magnetic bead detection using the magnetic sensor 220. The second magnetic-field applying mechanism 160 applies a magnetic field to magnetic beads 410 bound to the biomolecular label 400 or to molecules near the biomolecular label 400 during magnetic detection using the magnetic sensor 220. The second magnetic-field applying mechanism 160 can be used independently of the first magnetic-field applying mechanism 150 to apply a magnetic field suitable for magnetic bead detection using the magnetic sensor 220. It is desirable that the magnetic field applied by the second magnetic-field applying mechanism 160 be substantially uniform over the entire surface of the magnetic sensor 220. The second magnetic-field applying mechanism 160 may be any member having a mechanism or function that allows it to apply a magnetic field, such as a permanent magnet or coil (electromagnet). A coil (electromagnet) is preferably used since the applied magnetic field can be electrically controlled and the apparatus configuration can be simplified. It is desirable that the first magnetic-field applying mechanism 150 apply a more intense magnetic field over the surface of the magnetic sensor 220 than the second magnetic-field applying mechanism 160.

The electrical-signal converting section 170 is a section that converts the magnetic bead detection results received from the detection device 200 into electrical signals.

The display section 180 is a section that displays the electrical signals received from the electrical-signal converting section 170 as the presence and concentration of the biomolecular label.

### Detection Device

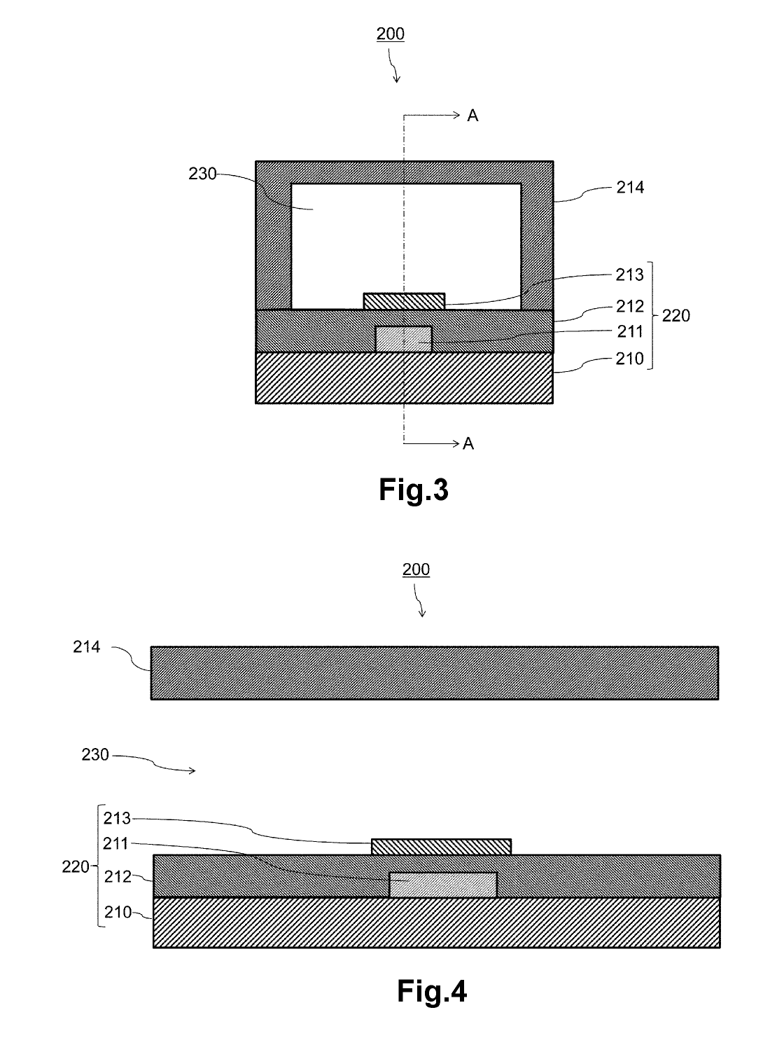

As shown in FIGS. 3 and 4, the detection device 200 according to the first embodiment includes the channel space 230, the magnetic sensor 220, and a channel member 214. The channel space 230 is a space defined by the wall surface of the channel member 214 and the surface (wall surface) of the magnetic sensor 220, described later. The liquid containing the biomolecular label 400 that has been supplied to the biologically-derived-liquid inlet 110 or the liquid containing the biomolecular label 400 that has been treated in the bodily-fluid chemical treatment section 13 and the liquid containing the magnetic beads 410 that has been supplied to the magnetic-bead containing liquid inlet 120 flow through the channel space 230. FIG. 3 is a sectional view of the detection device 200 taken in a plane perpendicular to the direction in which liquid flows through the channel space 230. FIG. 4 is a sectional view taken along line A-A in FIG. 3.

As shown in FIGS. 3 and 4, the magnetic sensor 220 includes a support 210, a magnetic sensing element 211, a protective layer 212, and an organic layer 213.

The support 210 is a support for imparting sufficient mechanical strength to handle the detection device 200 and is also a substrate for the fabrication of the magnetic sensing element 211. From the viewpoint of mechanical strength and the process of fabricating the magnetic sensing element 211, the protective layer 212, and the organic layer 213, it is preferred that the support 210 be formed of, for example, Si, SiO₂, ITO, glass, or Al₂O₃, most preferably Si, which is inexpensive.

The magnetic sensing element 211 is an element used for magnetic bead detection. An example of the magnetic sensing element 211 is a giant magnetoresistive (GMR) element. The magnetic sensing element 211 is formed on the support 210 by a technique such as vapor deposition.

The protective layer 212 is a layer for protecting the magnetic element 211 from the atmosphere. Noble metals, carbon, metal oxides, metal carbides, and metal nitrides, which are chemically stable, may be used, including Au, Pt, amorphous carbon, diamond-like carbon (DLC), SiO₂, Al₂O₃, TiO₂, ITO, SiC, and Si₃N₄. A suitable material is selected from the viewpoint of the process of forming the organic layer 213. The protective layer 212 is formed over the support 210 and the magnetic sensing element 211 by a technique such as vapor deposition. As shown in FIG. 4, the protective layer 212 is thinner over the magnetic sensing element 211 than over the remaining portion. It is preferred that there is little or no step on the top surface of the protective layer 212. Such a protective layer 212 can be formed by a lift-off process using a resist formed on the magnetic sensing element 211.

The organic layer 213 is a layer for immobilizing the biomolecular label 400 onto the surface of the magnetic sensor 220. The organic layer 213 has groups capable of binding to the biomolecular label 400 on the surface thereof. For example, the groups capable of binding to the biomolecular label 400 are carboxyl (—COOH) or amine (—NH₂) groups. A suitable organic material is selected as the organic material used to form the organic layer 213 depending on the groups capable of binding to the biomolecular label 400. For example, phosphonic acid or 3-aminopropyltriethoxysilane may be used. Nucleic acids and antibodies may also be used for the organic layer 213 for binding to the biomolecular label 400. The organic layer 213 is formed on the protective layer 212 by a technique such as evaporation, vapor deposition, solution immersion, or the Langmuir-Blodgett technique.

The magnetic sensor 220 allows the biomolecular label 400 to be immobilized onto the surface thereof and magnetically detects the biomolecular label 440. In the present invention, “the surface of the magnetic sensor” refers to the outermost surface of a layer formed on a magnetic sensing element, i.e., the surface onto which the biomolecular label is immobilized. In the first embodiment, the surface of the organic layer 213 corresponds to the surface of the magnetic sensor 220. The surface of the organic layer 213, serving as the surface of the magnetic sensor 220, forms a portion of the channel wall surface defining the channel space 230.

The channel member 214 is a member that is combined with the magnetic sensor 220 to define the channel space 230. The channel member 214 has a pair of side walls and a top surface joining the pair of side walls. The pair of side walls and the top surface define a rectangular channel. Examples of materials that may be used for the channel member 214 include glass, resin, and rubber, which are chemically stable. A suitable material is selected by taking into account, for example, the liquidity, flow rate, and viscosity of the liquid containing the biomolecular label 400 and the liquid containing the magnetic beads 410 and the sealing between the channel member 214 and the magnetic sensor 220.

The detection device 200 is inserted into the detection device insertion slot 190 such that the magnetic sensor 220 is located away from the first magnetic-field applying mechanism 150 in the detection device insertion slot 190.

### Biomolecular Label

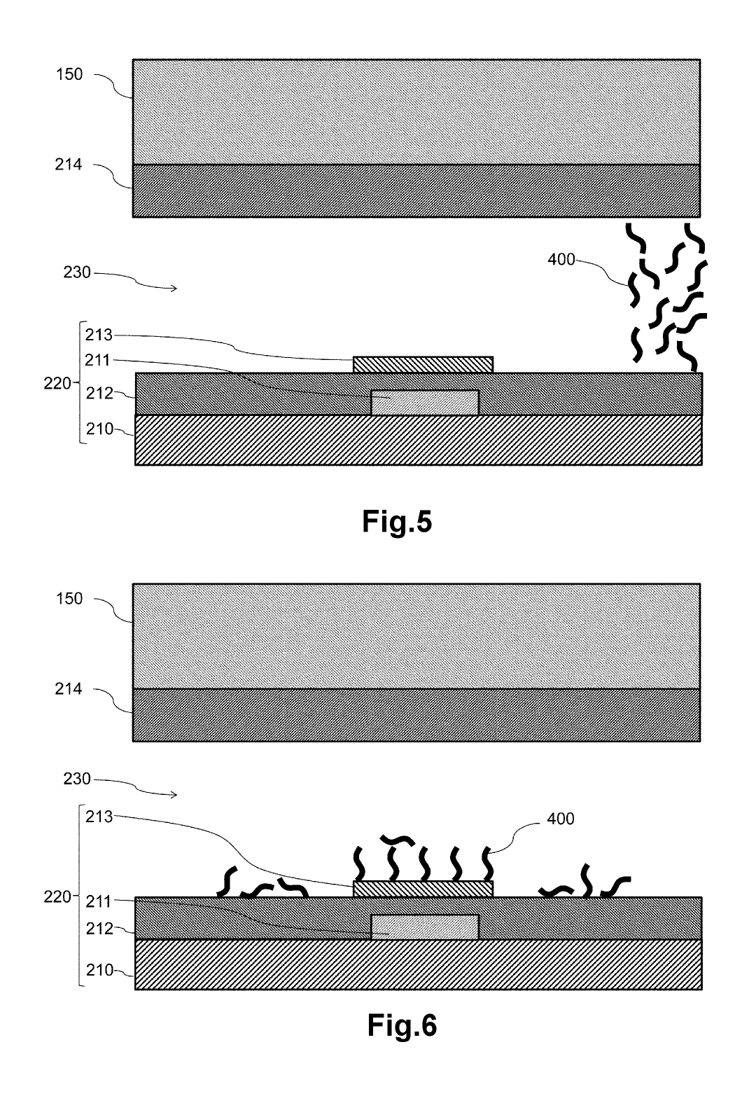

The biomolecular label 400 in the first embodiment shown in FIGS. 5 to 14 is, for example, a disease-related protein capable of protein-protein interaction, such as a receptor protein, adhesion protein, antigen, or antibody having a ligand capable of binding. Such substances include proteins that can be used for the diagnosis of diseases, such as those that indicate the presence of a disease as an increase or decrease in the amount of the substance present, including growth factors such as epidermal growth factor (EGF), platelet-derived growth factor (PDGF), brain-derived growth factor (BDGF), and vascular endothelial growth factor (VEGF); cell adhesion factors such as fibronectin, laminin, and vitronectin; hormones such as insulin, somatostatin, somatotropin, and gonadotropin-releasing hormone; lipoproteins such as LDL; and various analyte molecules such as tumor markers and antibodies. Viruses and bacteria such as HIV and HBV and analyte molecules such as nucleic acids such as oncogenes can also be used as biomolecular labels. In addition to the analyte molecules listed above, molecules bound to analyte molecules as listed above and molecules obtained by converting analyte molecules as listed above into labels can be used as biomolecular labels. The liquid containing the biomolecular label 400 needs to have the property of well dispersing the biomolecular label 400 without dissolving it. A buffer solution having a suitable pH is selected depending on the type of binding reaction between the magnetic beads 410 and the biomolecular label 400. For example, a buffer solution containing trishydroxymethylaminomethane and ethylenediaminetetraacetic acid and adjusted to a pH of 7 to 8 is used for the binding of biotin, serving as the biomolecular label 400, to streptavidin, serving as a surface substance on the magnetic beads 410.

### Magnetic Beads

The magnetic beads 410 shown in FIGS. 8 to 14, which are detected by the magnetic sensor 220, bind to the biomolecular label 400. The magnetic beads 410 have a structure including a magnetic material inside the magnetic beads 410. Examples of magnetic materials include ferromagnetic and superparamagnetic materials such as iron and iron oxide. The magnetic material inside the magnetic beads 410 is coated with an organic material capable of binding to the biomolecular label 400. Specific examples of such organic materials include those containing reactive groups such as amine or carboxyl groups. For example, streptavidin or hydroxyapatite can be used. These organic materials bind to biomolecular labels. A suitable organic material is selected depending on the disease to be identified, prevented, or diagnosed. The magnetic beads 410 have a suitable structure selected depending on the purpose, such as a structure including fine magnetic particles dispersed in the organic material or a structure including a magnetic core coated with the organic material. The magnetic beads 410 have a size of 10 nm to 100 μm. A suitable size is selected depending on the purpose.

### Method for Operating Detection System

A method for operating the detection system 300 (an example of a detection method according to the present invention) will now be described with reference to FIGS. 5 to 12. A biologically derived liquid is first supplied to the biologically-derived-liquid inlet 110. The biologically derived liquid supplied to the biologically-derived-liquid inlet 110 is optionally treated in the bodily-fluid chemical treatment section 130 and is passed through the channel space 230 as a liquid containing the biomolecular label 400. FIG. 5 shows the initial state in the step of pumping the liquid containing the biomolecular label 400 through the channel space 230 from right to left in the figure.

As shown in FIG. 6, pumping is then stopped. The liquid containing the biomolecular label 400 in the channel space 230 is left standing so that the biomolecular label 400 settles. Of the biomolecular label 400 that has settled, the biomolecular label 400 on the magnetic sensor 220 is immobilized onto the surface of the magnetic sensor 220 by the organic layer 213, whereas the remaining biomolecular label 400 is deposited on the protective layer 212 without being immobilized.

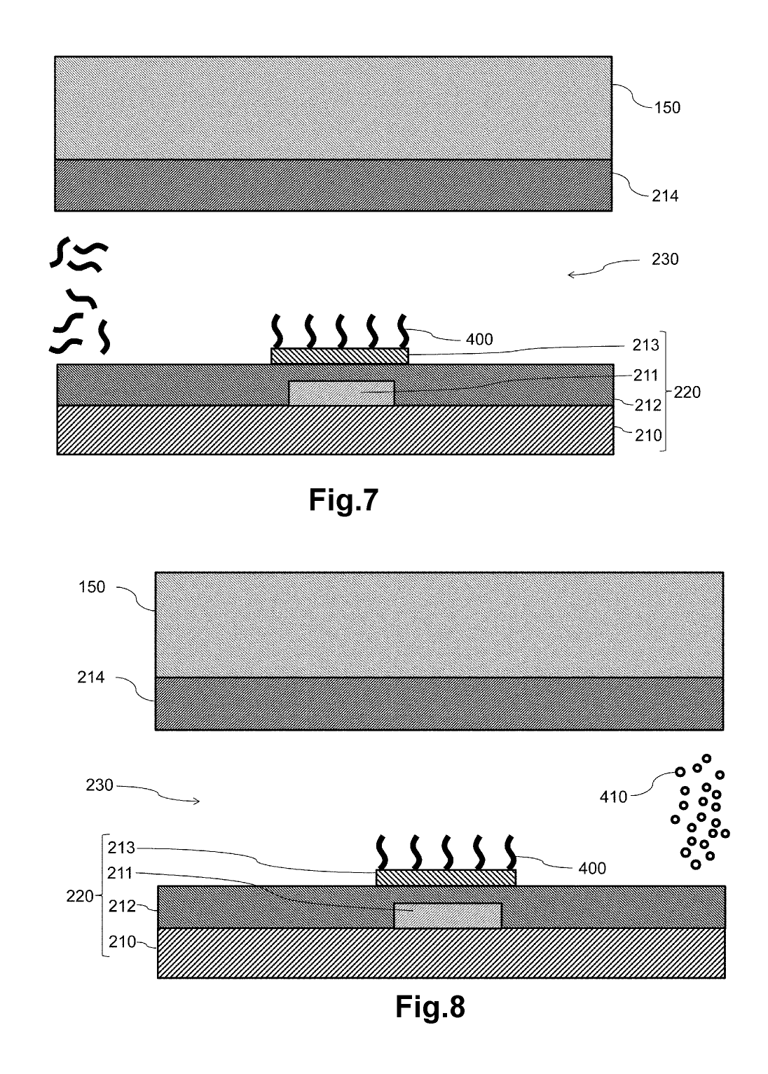

As shown in FIG. 7, the biomolecular label 400 deposited on the protective layer 212 without being immobilized is then removed by passing a liquid containing no biomolecular label through the channel space 230 from right to left in the figure. The liquid containing no biomolecular label is supplied from the magnetic bead inlet 120 or a dedicated inlet (not shown). This liquid is preferably the same as the buffer in which the biomolecular label 400 is dispersed.

A liquid containing the magnetic beads 410 is then supplied to the magnetic-bead containing liquid inlet 120. The liquid containing the magnetic beads 410 is passed through the channel space 230. FIG. 8 shows the initial state in the step of pumping the liquid containing the magnetic beads 410 through the channel space 230 from right to left in the figure.

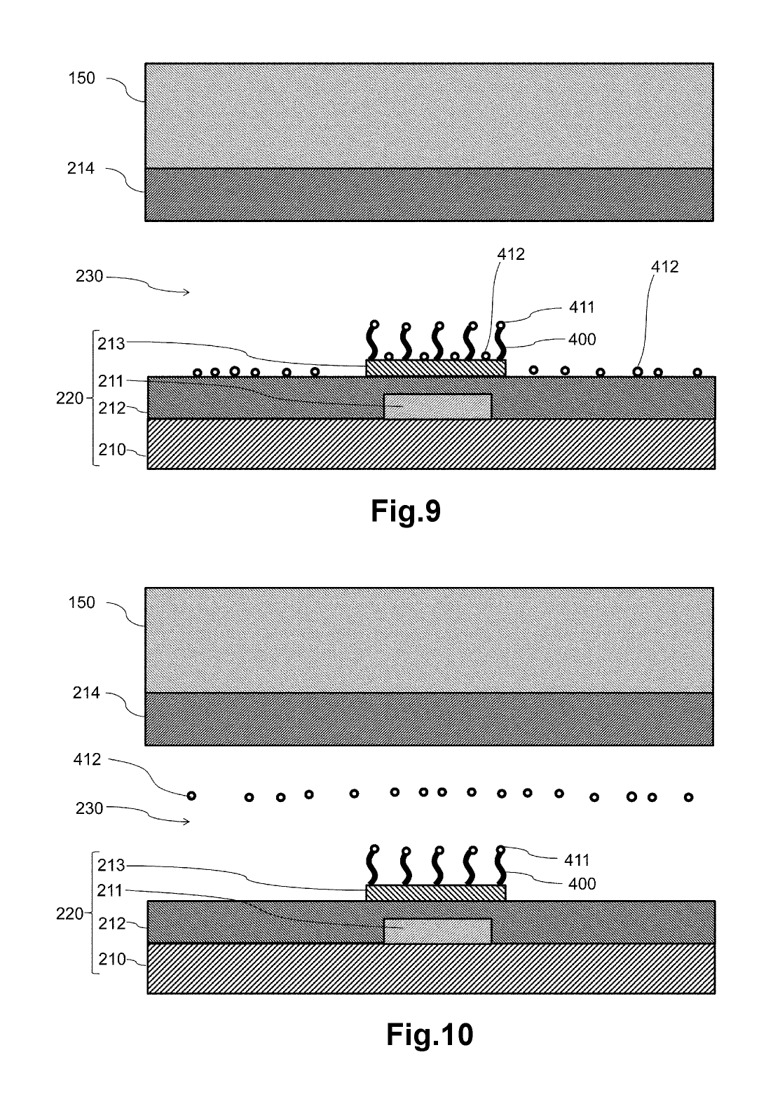

As shown in FIG. 9, pumping is then stopped. The liquid containing the magnetic beads 410 in the channel space 230 is left standing so that the magnetic beads 410 settle. Of the magnetic beads 410 that have settled, bound magnetic beads 411 are bound to the biomolecular label 400, whereas other nonspecifically adsorbed magnetic beads 412 are nonspecifically adsorbed mainly on the bottom surface of the channel space 230 (i.e., on the surface of the protective layer 212 and the surface of the organic layer 213), for example, by hydrophobic interactions or electrostatic interactions.

The first magnetic-field applying mechanism 150 is then used to apply a magnetic field in a direction in which the magnetic beads 410 (nonspecifically adsorbed magnetic beads 412) are moved away from the surface of the magnetic sensor 220. As shown in FIG. 10, the nonspecifically adsorbed magnetic beads 412, which have a relatively weak binding force, are attracted in the direction away from the surface of the magnetic sensor 220 by the magnetic field applied by the first magnetic-field applying mechanism 150, whereas the bound magnetic beads 411, which have a relatively strong binding force, remain bound to the biomolecular label 400 without being attracted by the magnetic field applied by the first magnetic-field applying mechanism 150. The intensity of the magnetic field applied by the first magnetic-field applying mechanism 150 is high enough for the nonspecifically adsorbed magnetic beads 412 to be attracted in the direction away from the surface of the magnetic sensor 220 and is low enough for the bound magnetic beads 411 to remain bound to the biomolecular label 400. If the first magnetic-field applying mechanism 150 is a coil, a magnetic field is applied from the first magnetic-field applying mechanism 150 by applying a current to the coil. If the first magnetic-field applying mechanism 150 is a permanent magnet, a magnetic field is applied from the first magnetic-field applying mechanism 150 by moving the permanent magnet toward the surface of the magnetic sensor 220.

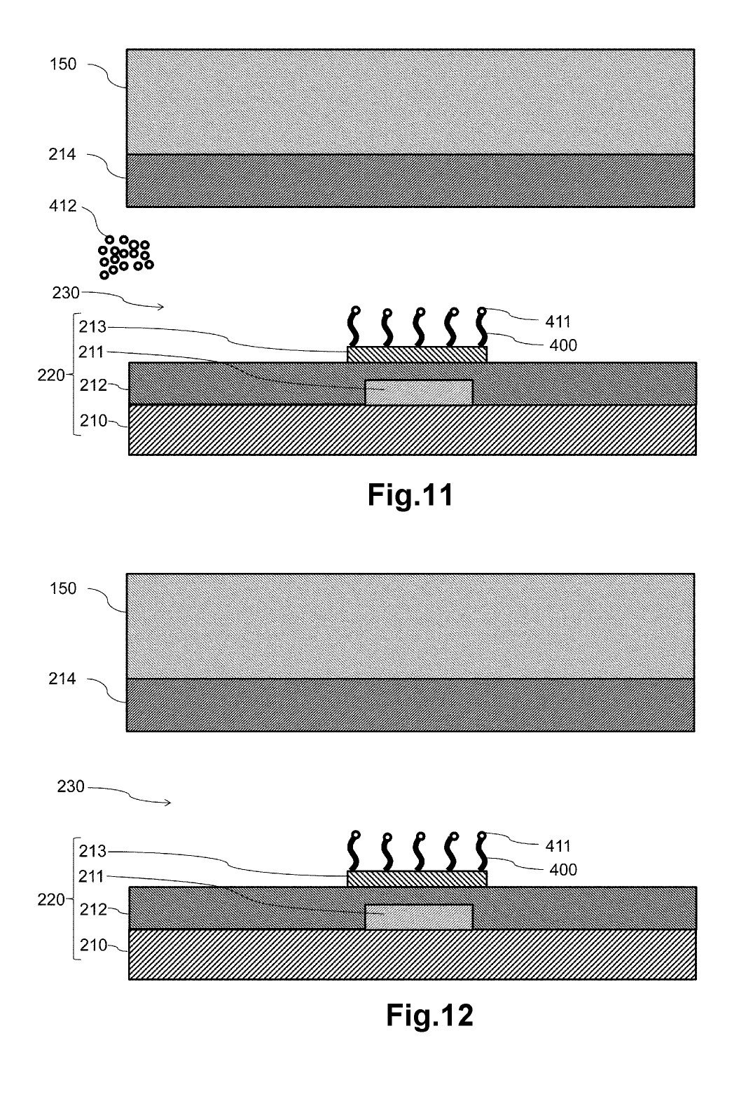

As shown in FIG. 11, a liquid containing no magnetic beads is then passed through the channel space 230 from right to left in the figure to remove the nonspecifically adsorbed magnetic beads 412 moved away from the surface of the magnetic sensor 220 while a magnetic field is being applied from the first magnetic-field applying mechanism 150. Alternatively, the liquid containing no magnetic beads may be passed through the channel space 230 to remove the nonspecifically adsorbed magnetic beads 412 moved away from the surface of the magnetic sensor 220 after the application of a magnetic field from the first magnetic-field applying mechanism 150 is stopped and before the nonspecifically adsorbed magnetic beads 412 moved away from the surface of the magnetic sensor 220 settle again on the surface of the magnetic sensor 220. The liquid containing no magnetic beads is supplied from the magnetic bead inlet 120 or a dedicated inlet (not shown). This liquid is preferably the same as the buffer in which the magnetic beads are dispersed. If the first magnetic-field applying mechanism 150 is a coil, the application of a magnetic field from the first magnetic-field applying mechanism 150 is stopped by stopping the application of a current to the coil. If the first magnetic-field applying mechanism 150 is a permanent magnet, the application of a magnetic field from the first magnetic-field applying mechanism 150 is stopped by moving the permanent magnet away from the surface of the magnetic sensor 220.

Thus, the liquid containing no magnetic beads is passed through the channel space 230 while a magnetic field is being applied from the first magnetic-field applying mechanism 150 or after the application of a magnetic field from the first magnetic-field applying mechanism 150 is stopped. This allows the nonspecifically adsorbed magnetic beads 412 to be more quickly removed from the surface of the magnetic sensor 220 than passing the liquid containing no magnetic beads through the channel space 230 without applying a magnetic field.

FIG. 12 shows the state after the removal of the nonspecifically adsorbed magnetic beads 412. Specifically, the magnetic beads 410 (bound magnetic beads 411) are present only on the biomolecular label 400 and are not present on the remaining portion. Thus, by measuring the magnetic field generated from the bound magnetic beads 411 using the magnetic sensor 220 (i.e., by performing magnetic detection using the magnetic sensor 220), the number or concentration of the biomolecular label 400 can be accurately measured.

A method for detecting a biomolecular label by magnetic detection using the magnetic sensor 220 will now be described. In the state in FIG. 12, the second magnetic-field applying mechanism 160 is used to apply a magnetic field to the bound magnetic beads 411. A magnetic field generated from the bound magnetic beads 411 in response to the applied magnetic field is detected and output as an electrical signal by the magnetic sensor 220. The electrical signal is fed through the electrical-signal converting section 170 to the display section 180. The value of the magnetic field is displayed as the number of the bound magnetic beads 411 or the number or concentration of the biomolecular label 400. If the second magnetic-field applying mechanism 160 is a coil, a magnetic field is applied from the second magnetic-field applying mechanism 160 by applying a current to the coil. If the second magnetic-field applying mechanism 160 is a permanent magnet, a magnetic field is applied from the second magnetic-field applying mechanism 160 by moving the permanent magnet toward the surface of the magnetic sensor 220.

Thus, in the detection system 300, the detection apparatus 100, and the method for operating the detection system 300 according to the first embodiment, the nonspecifically adsorbed magnetic beads 412 nonspecifically adsorbed on the surface of the magnetic sensor 220 without being bound to the biomolecular label 400 are moved away from the surface of the magnetic sensor 220 by the magnetic field applied by the first magnetic-field applying mechanism 150. This reduces the influence of the nonspecifically adsorbed magnetic beads 412 during magnetic bead detection using the magnetic sensor 220, thus permitting accurate detection of the bound magnetic beads 411 bound to the biomolecular label 400.

Furthermore, the detection system 300 and the detection apparatus 100 according to the first embodiment include the second magnetic-field applying mechanism 160, which applies a magnetic field to the bound magnetic beads 411 bound to the biomolecular label 400 during magnetic bead detection using the magnetic sensor 220. The second magnetic-field applying mechanism 160 can be used to apply a magnetic field suitable for magnetic bead detection using the magnetic sensor 220, thus permitting sensitive detection of the bound magnetic beads 411 bound to the biomolecular label 400.

Furthermore, in the method for operating the detection system 300 according to the first embodiment, a liquid containing no magnetic beads is passed through the channel space 230 while a magnetic field is being applied from the first magnetic-field applying mechanism 150 or after a magnetic field is applied from the first magnetic-field applying mechanism 150, and magnetic detection is then performed using the magnetic sensor 220. This allows the nonspecifically adsorbed magnetic beads 412 moved away from the surface of the magnetic sensor 220 to be removed from around the magnetic sensor 220, thus permitting more accurate detection.

Although the detection system 300 and the detection apparatus 100 according to the first embodiment described above include the second magnetic-field applying mechanism 160, the first magnetic-field applying mechanism 150 may be used to apply a magnetic field to the bound magnetic beads 411 bound to the biomolecular label 400 during magnetic detection using the magnetic sensor 220. In this case, the second magnetic-field applying mechanism 160 may be omitted.

In addition, in the method for operating the detection system 300 according to the first embodiment described above, a liquid containing no magnetic beads is passed through the channel space 230 while a magnetic field is being applied from the first magnetic-field applying mechanism 150 or after a magnetic field is applied from the first magnetic-field applying mechanism 150, and magnetic detection is then performed using the magnetic sensor 220; however, passing the liquid containing no magnetic beads through the channel space 230 may be omitted. In this case, a magnetic field may be applied in the direction in which the magnetic beads 410 are moved away from the surface of the magnetic sensor 220, and magnetic detection may then be performed using the magnetic sensor 220. This reduces the influence of the nonspecifically adsorbed magnetic beads 412, thus permitting accurate detection of the bound magnetic beads 411 bound to the biomolecular label 400.

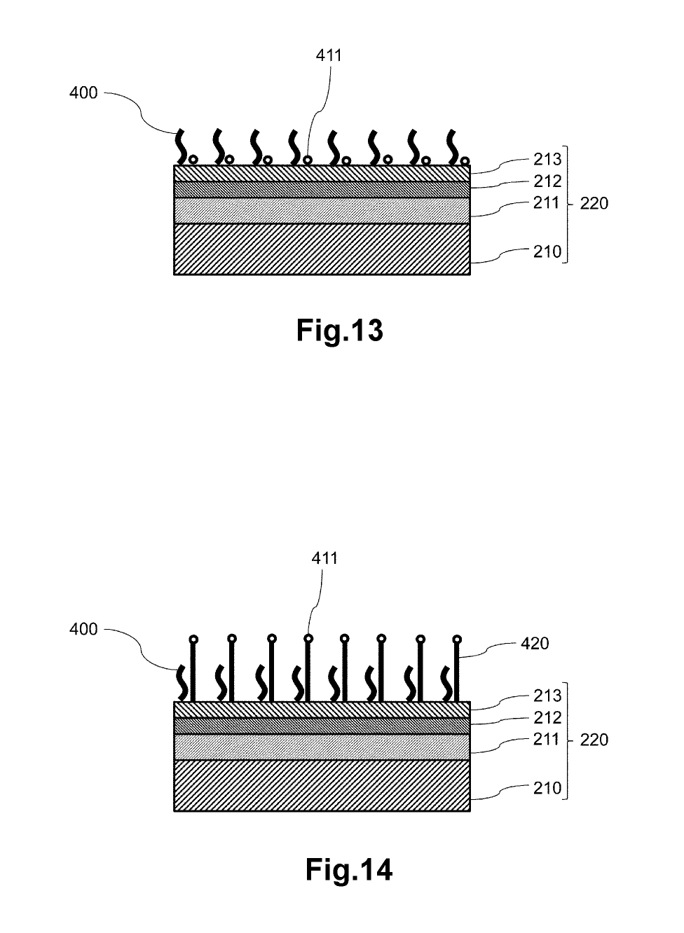

In addition, although the embodiment described above has been described with reference to an example in which the magnetic beads 410 bind directly to the biomolecular label 400, as shown in FIG. 13, the magnetic beads 410 may be provided near the biomolecular label 400 without being directly bound to the biomolecular label 400 by selectively binding the magnetic beads 410 to molecules near the biomolecular label 400 (i.e., molecules present on the surface of the magnetic sensor 220 near the biomolecular label 400), for example, by supporting a catalyst (not shown) on the biomolecular label 400.

Alternatively, as shown in FIG. 14, the magnetic beads 410 may be provided near the biomolecular label 400 without being directly bound to the biomolecular label 400 by selectively growing magnetic-bead supporting molecules 420 near the biomolecular label 400 and binding the magnetic beads 410 to the magnetic-bead supporting molecules 420, for example, by supporting a catalyst (not shown) on the biomolecular label 400.

### Second Embodiment

As for a second embodiment, differences from the first embodiment will be mainly described, and common details are not described where appropriate.

### Detection Apparatus

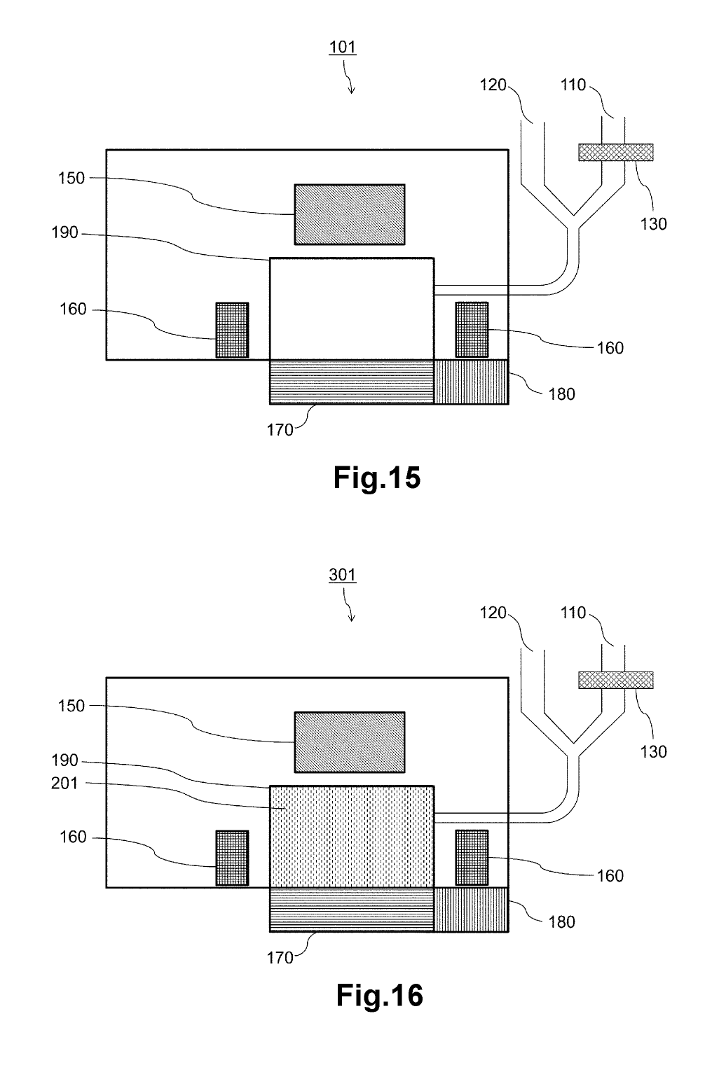

As shown in FIG. 15, a detection apparatus 101 according to the second embodiment is the detection apparatus 100 according to the first embodiment having the liquid outlet 140 removed therefrom. The liquids supplied to the biologically-derived-liquid inlet 110 and the magnetic-bead containing liquid inlet 120 of the detection apparatus 101 are introduced into a well space 500 in a detection device 201, described later. The remaining configuration of the detection apparatus 101 is identical to that of the detection apparatus 100 according to the first embodiment.

### Detection System

As shown in FIG. 16, a detection system 301 according to the second embodiment includes the detection apparatus 101 instead of the detection apparatus 100 according to the first embodiment and the detection device 201 instead of the detection device 200 according to the first embodiment. The detection system 301 operates with the detection device 201 inserted in the detection device insertion slot 190 of the detection apparatus 101.

### Detection Device

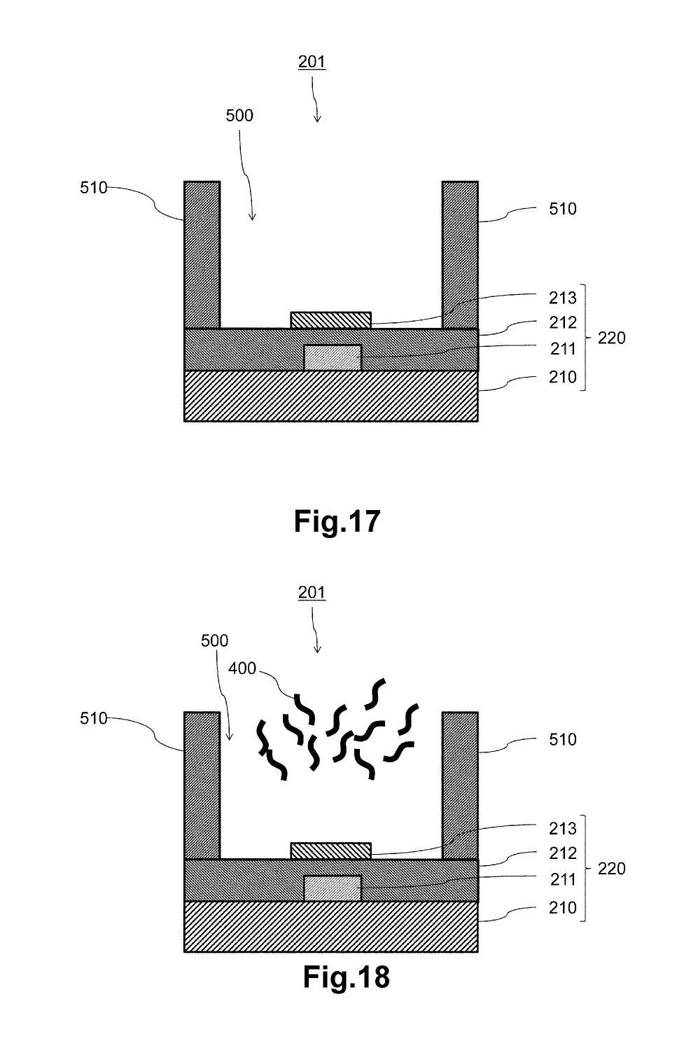

As shown in FIG. 17, the detection device 201 according to the second embodiment includes the well space 500, which is an open-top well-like space, the magnetic sensor 220, and a well member 510. The well space 500 is a space defined by the wall surface of the well member 510 and the surface (wall surface) of the magnetic sensor 220. The liquid containing the biomolecular label 400 that has been supplied to the biologically-derived-liquid inlet 110 or the liquid containing the biomolecular label 400 that has been treated in the bodily-fluid chemical treatment section 130 and the liquid containing the magnetic beads 410 that has been supplied to the magnetic-bead containing liquid inlet 120 are introduced into the well space 500. FIG. 17 is a sectional view of the detection device 201.

The detection device 201 according to the second embodiment has the same configuration as the detection device 200 according to the first embodiment except that the channel space 230 is replaced by the well space 500 and the channel member 214 is represented by the well member 510.

In the first embodiment, the biomolecular label 400 is immobilized onto the surface of the magnetic sensor 220 after the liquid containing the biomolecular label 400 is passed through the channel space 230, and the magnetic beads 410 are bound to the biomolecular label 400 or to molecules near the biomolecular label 400 after the liquid containing the magnetic beads 410 is passed through the channel space 230. In the second embodiment, these liquids are introduced into the well space 500 and are then retained therein to immobilize the biomolecular label 400 and to bind the magnetic beads.

The well member 510 is a member for defining the well space 500 and forms the side wall surface of the well space 500. Examples of materials that may be used for the well member 510 include glass, resin, and rubber, which are chemically stable. A suitable material is selected by taking into account, for example, the liquidity and viscosity of the liquid containing the biomolecular label 400 and the liquid containing the magnetic beads 410 and the sealing between the well member 510 and the magnetic sensor 220.

### Method for Operating Detection System

A method for operating the detection system 301 (an example of a detection method according to the present invention) will now be described with reference to FIGS. 18 to 26. A biologically derived liquid is first supplied to the biologically-derived-liquid inlet 110. The biologically derived liquid supplied to the biologically-derived-liquid inlet 110 is optionally treated in the bodily-fluid chemical treatment section 130 and is introduced into the well space 500 as a liquid containing the biomolecular label 400. FIG. 18 shows the initial state in the step of introducing the liquid containing the biomolecular label 400 into the well space 500 from top to bottom in the figure.

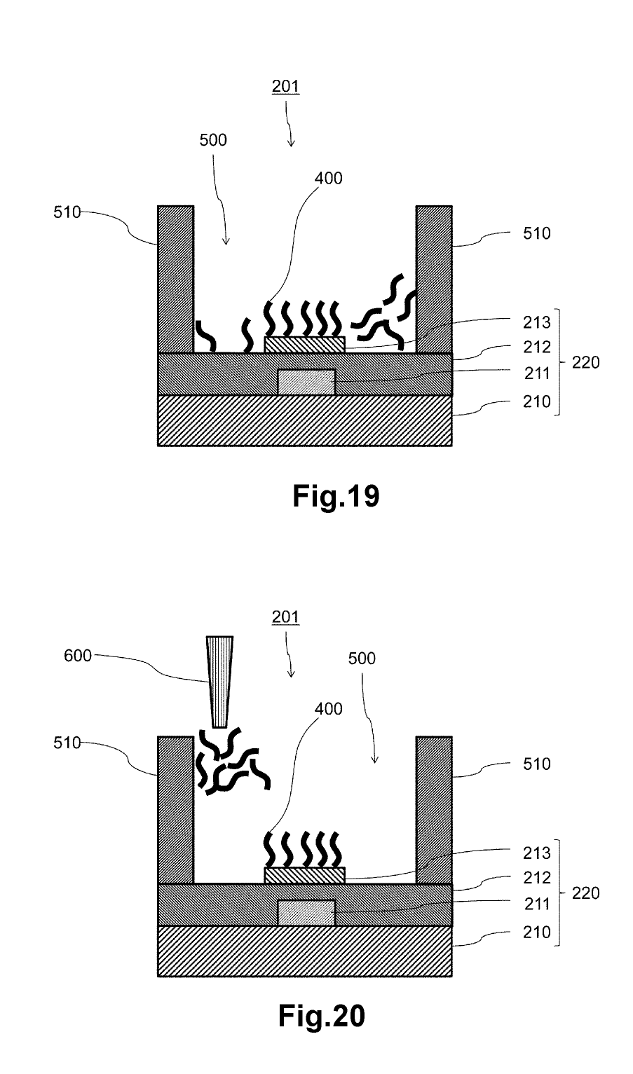

As shown in FIG. 19, the introduction of the liquid containing the biomolecular label 400 is then stopped. The liquid containing the biomolecular label 400 is retained in the well space 500 for a predetermined period of time so that the biomolecular label 400 settles. Of the biomolecular label 400 that has settled, the biomolecular label 400 on the magnetic sensor 220 is immobilized onto the surface of the magnetic sensor 220 by the organic layer 213, whereas the remaining biomolecular label 400 is deposited on the protective layer 212 without being immobilized.

As shown in FIG. 20, the biomolecular label 400 deposited on the protective layer 212 is then removed from the top of the well space 500, for example, using a dropper 600.

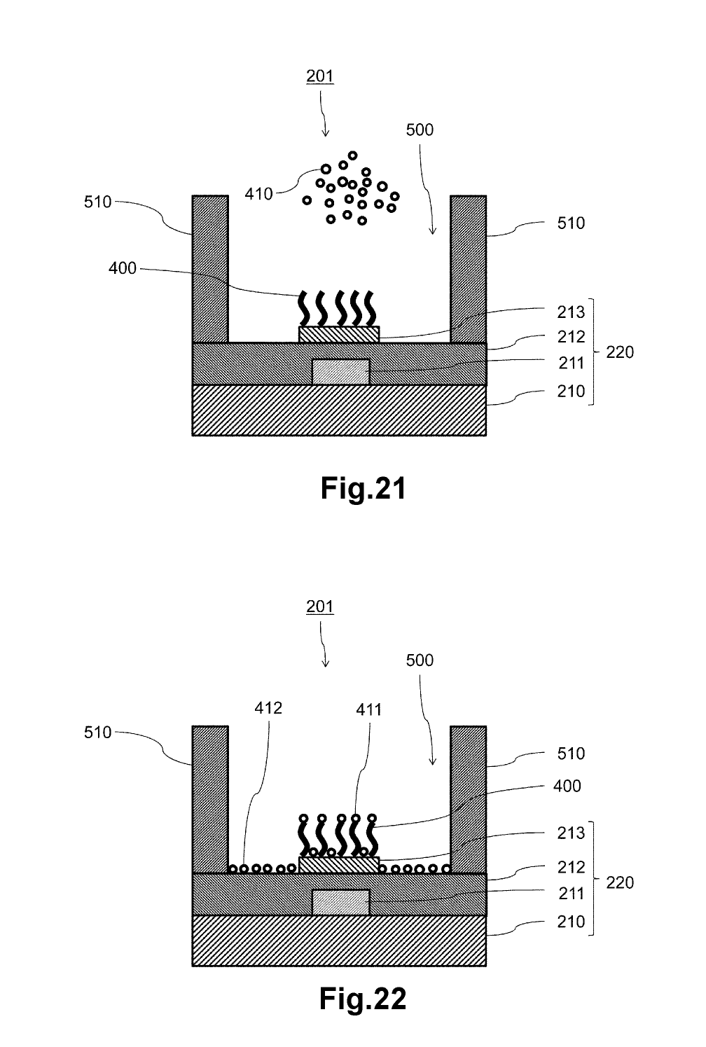

A liquid containing the magnetic beads 410 is then supplied to the magnetic-bead containing liquid inlet 120. As shown in FIG. 21, the liquid containing the magnetic beads 410 is introduced into the well space 500. FIG. 21 shows the initial state in the step of introducing the liquid containing the magnetic beads 410 into the well space 500 from top to bottom in the figure.

As shown in FIG. 22, the introduction of the liquid containing the magnetic beads 410 is then stopped. The liquid containing the magnetic beads 410 is retained in the well space 500 for a predetermined period of time so that the magnetic beads 410 settle. Of the magnetic beads 410 that have settled, bound magnetic beads 411 are bound to the biomolecular label 400, whereas other nonspecifically adsorbed magnetic beads 412 are nonspecifically adsorbed mainly on the bottom surface of the well space 500 (i.e., on the surface of the protective layer 212 and the surface of the organic layer 213), for example, by hydrophobic interactions or electrostatic interactions.

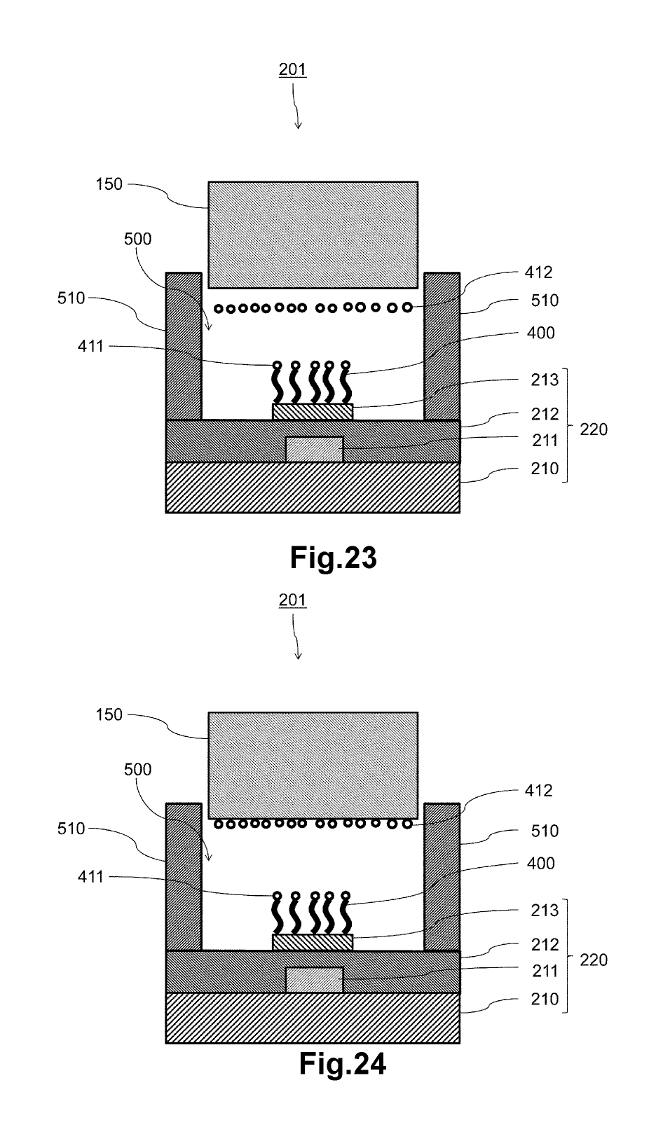

As shown in FIG. 23, the first magnetic-field applying mechanism 150 is then used to apply a magnetic field in a direction in which the magnetic beads 410 (nonspecifically adsorbed magnetic beads 412) are moved away from the surface of the magnetic sensor 220. As shown in FIG. 23, the nonspecifically adsorbed magnetic beads 412, which have a relatively weak binding force, are attracted in the direction away from the surface of the magnetic sensor 220 by the magnetic field applied by the first magnetic-field applying mechanism 150, whereas the bound magnetic beads 411, which have a relatively strong binding force, remain bound to the biomolecular label 400 without being attracted by the magnetic field applied by the first magnetic-field applying mechanism 150. The intensity of the magnetic field applied from the first magnetic-field applying mechanism 150 is high enough for the nonspecifically adsorbed magnetic beads 412 to be attracted in the direction away from the surface of the magnetic sensor 220 and is low enough for the bound magnetic beads 411 to remain bound to the biomolecular label 400. If the first magnetic-field applying mechanism 150 is a coil, a magnetic field is applied from the first magnetic-field applying mechanism 150 by applying a current to the coil. If the first magnetic-field applying mechanism 150 is a permanent magnet, a magnetic field is applied from the first magnetic-field applying mechanism 150 by moving the permanent magnet toward the surface of the magnetic sensor 220.

As shown in FIG. 24, the nonspecifically adsorbed magnetic beads 412 are then caused to become attached to the first magnetic-field applying mechanism 150. It is desirable that the first magnetic-field applying mechanism 150 having the nonspecifically adsorbed magnetic beads 412 attached thereto be moved to a position away from the well space 500. By causing the nonspecifically adsorbed magnetic beads 412 to become attached to the first magnetic-field applying mechanism 150, the nonspecifically adsorbed magnetic beads 412 moved away from the surface of the magnetic sensor 220 can be removed from around the magnetic sensor 220, thus permitting more accurate detection.

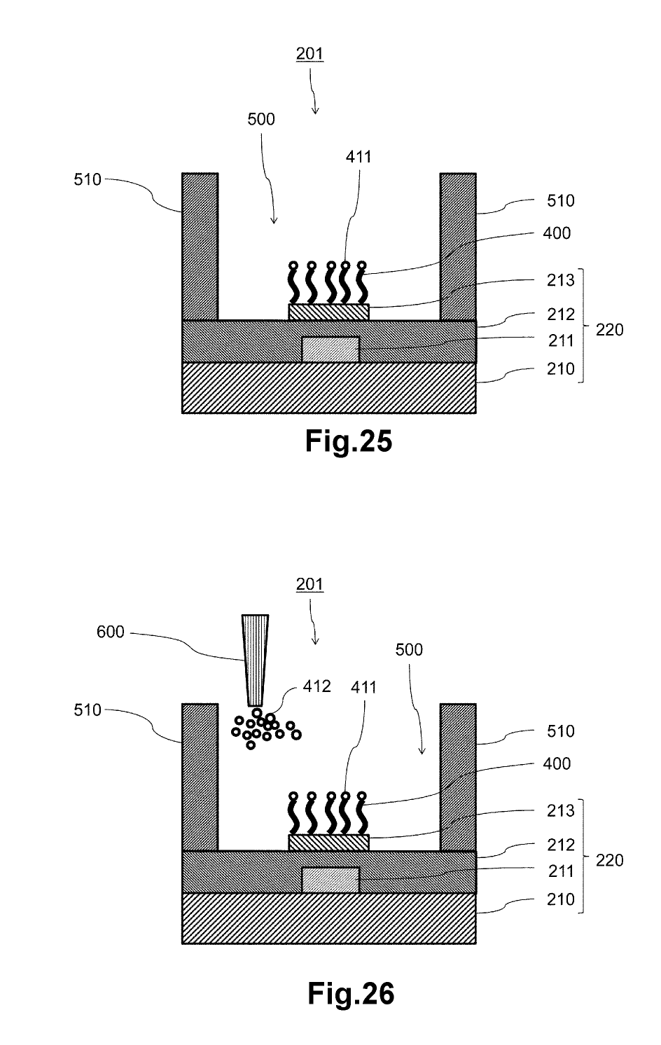

FIG. 25 shows the state after the removal of the nonspecifically adsorbed magnetic beads 412. Specifically, the magnetic beads 410 (bound magnetic beads 411) are present only on the biomolecular label 400 and are not present on the remaining portion. Thus, by measuring the magnetic field generated from the bound magnetic beads 411 using the magnetic sensor 220 (i.e., by performing magnetic detection using the magnetic sensor 220), the number or concentration of the biomolecular label 400 can be accurately measured.

A method for detecting a biomolecular label by magnetic detection using the magnetic sensor 220 will now be described. In the state in FIG. 25, as in the first embodiment, the second magnetic-field applying mechanism 160 is used to apply a magnetic field to the bound magnetic beads 411. A magnetic field generated from the bound magnetic beads 411 in response to the applied magnetic field is detected and output as an electrical signal by the magnetic sensor 220. The electrical signal is fed through the electrical-signal converting section 170 to the display section 180. The value of the magnetic field is displayed as the number of the bound magnetic beads 411 or the number or concentration of the biomolecular label 400. If the second magnetic-field applying mechanism 160 is a coil, a magnetic field is applied from the second magnetic-field applying mechanism 160 by applying a current to the coil. If the second magnetic-field applying mechanism 160 is a permanent magnet, a magnetic field is applied from the second magnetic-field applying mechanism 160 by moving the permanent magnet toward the surface of the magnetic sensor 220.

Thus, in the detection system 301, the detection apparatus 101, and the method for operating the detection system 301 according to the second embodiment, the nonspecifically adsorbed magnetic beads 412 nonspecifically adsorbed on the surface of the magnetic sensor 220 without being bound to the biomolecular label 400 are moved away from the surface of the magnetic sensor 220 by the magnetic field applied by the first magnetic-field applying mechanism 150. This reduces the influence of the nonspecifically adsorbed magnetic beads 412 during magnetic bead detection using the magnetic sensor 220, thus permitting accurate detection of the bound magnetic beads 411 bound to the biomolecular label 400.

Furthermore, the detection system 301 and the detection apparatus 101 according to the second embodiment include the second magnetic-field applying mechanism 160, which applies a magnetic field to the bound magnetic beads 411 bound to the biomolecular label 400 during magnetic bead detection using the magnetic sensor 220. The second magnetic-field applying mechanism 160 can be used to apply a magnetic field suitable for magnetic bead detection using the magnetic sensor 220, thus permitting sensitive detection of the bound magnetic beads 411 bound to the biomolecular label 400.

Furthermore, in the method for operating the detection system 301 according to the second embodiment, the liquid containing the biomolecular label 400 is introduced into the well space 500, in which the surface of the magnetic sensor 220 forms a portion of the wall surface thereof, to immobilize the biomolecular label 400 onto the surface of the magnetic sensor 220, the liquid containing the magnetic beads 410 is introduced into the well space 500 to bind at least some of the magnetic beads 410 to the biomolecular label 400 immobilized on the surface of the magnetic sensor 220 or to molecules near the biomolecular label 400 immobilized on the surface of the magnetic sensor 220, a magnetic field is applied to cause the magnetic beads 410 (nonspecifically adsorbed magnetic beads 412) to become attached to the first magnetic-field applying mechanism 150, which applies the magnetic field, and magnetic detection is then performed using the magnetic sensor 220. Thus, by causing the nonspecifically adsorbed magnetic beads 412 to become attached to the first magnetic-field applying mechanism 150, the nonspecifically adsorbed magnetic beads 410 moved away from the surface of the magnetic sensor 220 can be removed from around the magnetic sensor 220, thus permitting more accurate detection.

Although the detection system 301 and the detection apparatus 101 according to the second embodiment described above include the second magnetic-field applying mechanism 160, the first magnetic-field applying mechanism 150 may be used to apply a magnetic field to the bound magnetic beads 411 bound to the biomolecular label 400 during magnetic detection using the magnetic sensor 220. In this case, the second magnetic-field applying mechanism 160 may be omitted.

In addition, although the method for operating the detection system 301 according to the second embodiment described above has been described with reference to an example in which the nonspecifically adsorbed magnetic beads 412 are removed by causing the nonspecifically adsorbed magnetic beads 412 to become attached to the first magnetic-field applying mechanism 150, as shown in FIG. 26, the nonspecifically adsorbed magnetic beads 412 may be removed from the top of the well space 500, for example, using the dropper 600, while a magnetic field is being applied from the first magnetic-field applying mechanism 150. Alternatively, the nonspecifically adsorbed magnetic beads 412 may be removed from the top of the well space 500, for example, using the dropper 600, after the application of a magnetic field from the first magnetic-field applying mechanism 150 is stopped and before the nonspecifically adsorbed magnetic beads 412 moved away from the surface of the magnetic sensor 220 settle again on the surface of the magnetic sensor 220.

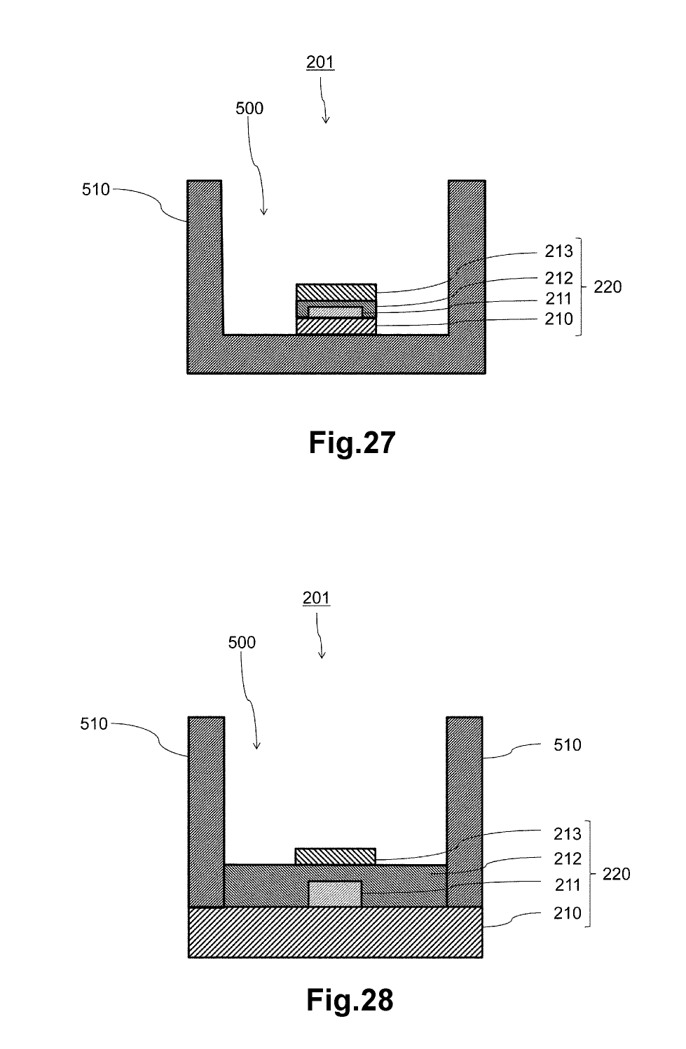

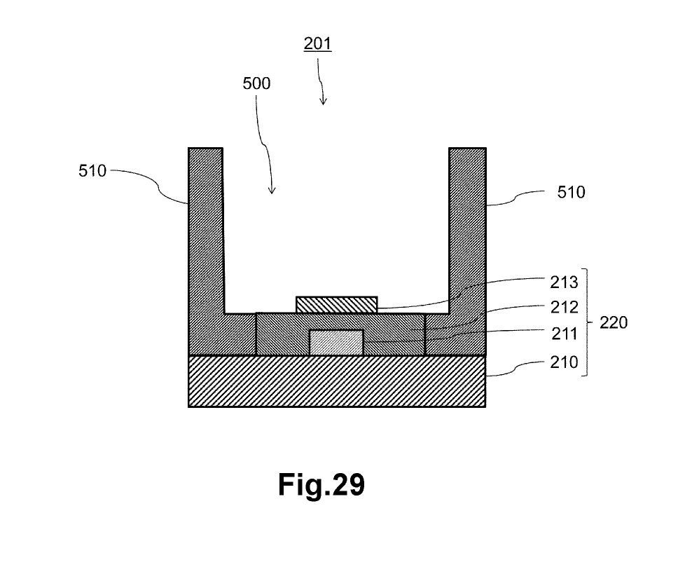

FIGS. 27 to 29 show example configurations as modifications of the detection device 201 according to the second embodiment. FIG. 27 shows an example in which the well member 510 forms the side wall surface and a portion of the bottom wall surface of the well space 500, with the magnetic sensor 220 placed on the bottom of the well member 510. FIG. 28 shows an example in which the well member 510 is placed such that a portion of the well member 510 faces the side surface of the protective layer 212. FIG. 29 shows an example in which a portion of the well member 510 forms a portion of the bottom wall surface of the well space 500.

After the biomolecular label 400 is immobilized onto the surface of the magnetic sensor 220 in the well space 500, as described in the second embodiment, the magnetic sensor 220 may be separated from the well member 510 and may be combined with the channel member 214 described in the first embodiment to define the channel space 230. The magnetic beads 410 may then be bound to the biomolecular label 400 or to molecules near the biomolecular label 400 in the channel space 230, as described in the first embodiment. That is, in this case, the channel space 230 serves as a space into which the liquid containing the magnetic beads 410 is introduced, and the biomolecular label 400 is immobilized on the surface of the magnetic sensor 220 forming a portion of the wall surface defining the channel space 230.

In addition, after the biomolecular label 400 is immobilized onto the surface of the magnetic sensor 220 in the channel space 230, as described in the first embodiment, the magnetic sensor 220 may be separated from the channel member 214 and may be combined with the well member 510 described in the second embodiment to define the well space 500. The magnetic beads 410 may then be bound to the biomolecular label 400 or to molecules near the biomolecular label 400 in the well space 500, as described in the second embodiment. That is, in this case, the well space 500 serves as a space into which the liquid containing the magnetic beads 410 is introduced, and the biomolecular label 400 is immobilized on the surface of the magnetic sensor 220 forming a portion of the wall surface defining the well space 500.

### REFERENCE SIGNS LIST

100 and 101 detection apparatus
110 biologically-derived-liquid inlet
120 magnetic-bead containing liquid inlet
130 bodily-fluid chemical treatment section
140 liquid outlet
150 first magnetic-field applying mechanism
160 second magnetic-field applying mechanism
170 electrical-signal converting section
180 display section
190 detection device insertion slot
200 and 201 detection device
210 support
211 magnetic sensing element
212 protective layer
213 organic layer
214 channel member
220 magnetic sensor
230 channel space
300 and 301 detection system
400 biomolecular label
410 magnetic bead
411 bound magnetic bead
412 nonspecifically adsorbed magnetic bead
420 magnetic-bead supporting molecule
500 well space
510 well member
600 dropper

1. A detection system comprising:
a detection device including a space, defined by a wall surface, into which a liquid containing magnetic beads is introduced and a magnetic sensor having a surface, forming a portion of the wall surface, onto which a biomolecular label is immobilized, wherein at least some of the magnetic beads bind to the biomolecular label or to a molecule near the biomolecular label; and
a first magnetic-field applying mechanism that applies a magnetic field in a direction in which the magnetic beads are moved away from the surface of the magnetic sensor.

2. A detection system comprising:
a detection device including a space, defined by a wall surface, into which a liquid containing a biomolecular label and a liquid containing magnetic beads are introduced and a magnetic sensor having a surface forming a portion of the wall surface, wherein at least some of the magnetic beads bind to the biomolecular label immobilized on the surface of the magnetic sensor or to a molecule near the biomolecular label immobilized on the surface of the magnetic sensor; and
a first magnetic-field applying mechanism that applies a magnetic field in a direction in which the magnetic beads are moved away from the surface of the magnetic sensor.

3. The detection system according to claim 1, further comprising a second magnetic-field applying mechanism that applies a magnetic field to the magnetic beads bound to the biomolecular label or to the molecule near the biomolecular label during magnetic detection using the magnetic sensor.

4. The detection system according to claim 1, wherein the space is a channel space through which the liquid containing the magnetic beads flows, wherein the surface of the magnetic sensor forms a portion of a channel wall surface defining the channel space.

5. The detection system according to claim 2, wherein the space is a channel space through which the liquid containing the biomolecular label and the liquid containing the magnetic beads flow, wherein the surface of the magnetic sensor forms a portion of a channel wall surface defining the channel space.

6. A detection apparatus comprising:
an insertion section into which a detection device is inserted, the detection device including a space, defined by a wall surface, into which a liquid containing magnetic beads is introduced and a magnetic sensor having a surface, forming a portion of the wall surface, onto which a biomolecular label is immobilized, wherein at least some of the magnetic beads bind to the biomolecular label or to a molecule near the biomolecular label; and
a first magnetic-field applying mechanism that applies a magnetic field in a direction in which the magnetic beads are moved away from the surface of the magnetic sensor.

7. A detection apparatus comprising:
an insertion section into which a detection device is inserted, the detection device including a space, defined by a wall surface, into which a liquid containing a biomolecular label and a liquid containing magnetic beads are introduced and a magnetic sensor having a surface forming a portion of the wall surface, wherein at least some of the magnetic beads bind to the biomolecular label immobilized on the surface of the magnetic sensor or to a molecule near the biomolecular label immobilized on the surface of the magnetic sensor; and
a first magnetic-field applying mechanism that applies a magnetic field in a direction in which the magnetic beads are moved away from the surface of the magnetic sensor.

8. The detection apparatus according to claim 6, further comprising a second magnetic-field applying mechanism that applies a magnetic field to the magnetic beads bound to the biomolecular label or to the molecule near the biomolecular label during magnetic detection using the magnetic sensor.

9. The detection apparatus according to claim 6, wherein the space is a channel space through which the liquid containing the magnetic beads flows, wherein the surface of the magnetic sensor forms a portion of a channel wall surface defining the channel space.

10. The detection apparatus according to claim 7, wherein the space is a channel space through which the liquid containing the biomolecular label and the liquid containing the magnetic beads flow, wherein the surface of the magnetic sensor forms a portion of a channel wall surface defining the channel space.

11. A detection method comprising bringing a liquid containing a biomolecular label into contact with a surface of a magnetic sensor to immobilize the biomolecular label onto the surface of the magnetic sensor; bringing a liquid containing magnetic beads into contact with the surface of the magnetic sensor to bind at least some of the magnetic beads to the biomolecular label immobilized on the surface of the magnetic sensor or to a molecule near the biomolecular label immobilized on the surface of the magnetic sensor; applying a magnetic field in a direction in which the magnetic beads are moved away from the surface of the magnetic sensor; and then performing magnetic detection using the magnetic sensor.

12. The detection method according to claim 11, wherein the liquid containing the biomolecular label is passed through a channel space in which the surface of the magnetic sensor forms a portion of a channel wall surface thereof to immobilize the biomolecular label onto the surface of the magnetic sensor, and the liquid containing the magnetic beads is passed through the channel space to bind at least some of the magnetic beads to the biomolecular label immobilized on the surface of the magnetic sensor or to a molecule near the biomolecular label immobilized on the surface of the magnetic sensor.

13. The detection method according to claim 12, wherein a liquid containing no magnetic beads is passed through the channel space while the magnetic field is being applied or after the application of the magnetic field is stopped, and magnetic detection is then performed using the magnetic sensor.

14. The detection method according to claim 11, wherein the liquid containing the biomolecular label is introduced into a well space in which the surface of the magnetic sensor forms a portion of a wall surface thereof to immobilize the biomolecular label onto the surface of the magnetic sensor, the liquid containing the magnetic beads is introduced into the well space to bind at least some of the magnetic beads to the biomolecular label immobilized on the surface of the magnetic sensor or to a molecule near the biomolecular label immobilized on the surface of the magnetic sensor, the magnetic field is applied to cause the magnetic beads to become attached to a magnetic-field applying mechanism that applies the magnetic field, and magnetic detection is then performed using the magnetic sensor.

15. The detection system according to claim 2, further comprising a second magnetic-field applying mechanism that applies a magnetic field to the magnetic beads bound to the biomolecular label or to the molecule near the biomolecular label during magnetic detection using the magnetic sensor.

16. The detection apparatus according to claim 7, further comprising a second magnetic-field applying mechanism that applies a magnetic field to the magnetic beads bound to the biomolecular label or to the molecule near the biomolecular label during magnetic detection using the magnetic sensor.
